(19) United States
(12) Patent Application Publication
(10) Pub. No.: US 2021/0322393 A1
(43) Pub. Date: Oct. 21, 2021

(54) METHODS OF TREATING COGNITIVE IMPAIRMENT ASSOCIATED WITH NEURODEGENERATIVE DISEASE

(71) Applicant: Aptinyx Inc., Evanston, IL (US)

(72) Inventors: Torsten M. Madsen, Evanston, IL (US); Ferenc Martenyi, Evanston, IL (US)

(21) Appl. No.: 17/110,481

(22) Filed: Dec. 3, 2020

Related U.S. Application Data
(60) Provisional application No. 62/943,430, filed on Dec. 4, 2019.

(51) Int. Cl. A61K 31/438

(52) U.S. Cl. CPC A61K 31/438

(57) ABSTRACT

Provided herein are methods of treating cognitive impairment associated with neurodegenerative disease in a patient by administering to the patient an effective amount of the Compound, or a pharmaceutically acceptable salt thereof.

### CROSS-REFERENCE TO RELATED APPLICATION

This application claims the benefit of the filing date of U.S. Provisional Application No. 62/943,430, filed Dec. 4, 2019, the entire content of which is hereby incorporated by reference herein.

### BACKGROUND

Parkinson's disease involves both motor and non-motor symptoms. Cognitive impairment in Parkinson's disease can be a severe disease feature with limited treatment options. A broad spectrum of cognitive deficits is associated with Parkinson's disease, from subjective cognitive decline to mild cognitive impairment (MCI-PD) and dementia (PDD). Mild cognitive impairment is common in nondemented Parkinson's disease patients and may be a precursor form of dementia. People with MCI-PD and PDD often report slower thinking and information processing. Attention, working memory, executive function, and visual-spatial function are the most frequently affected cognitive domains. Subjective or caregiver reported cognitive impairment might be misrepresented in patients with Parkinson's disease, therefore, an accurate diagnosis of MCI-PD requires neuropsychological tests that can reliably confirm cognitive decline.

The progression of Parkinson's disease symptoms can vary among patients due to the underlying pathology of the disease; however, cognitive dysfunction tends to be an early non-motor symptom, with 15 to 25% of newly diagnosed patients meeting MCI-PD classification and 75% progressing to PDD by 10 years post-diagnosis of Parkinson's disease. Longitudinal studies have shown that MCI-PD can progress to dementia leading to the diagnosis of PDD. Patients with MCI-PD are 6 times more likely than age-matched controls to develop dementia. This highlights the need for effective therapies in treating the cognitive impairments associated with Parkinson's disease.

Current treatment options for cognitive impairment with Parkinson's disease are limited. The cholinesterase inhibitor rivastigmine has been approved by regulators as the only treatment for PDD. There is no treatment for earlier forms of cognitive impairment and no successful treatments for MCI-PD have been identified in placebo-controlled, randomized trials.

Parkinson's disease involves changes to dopaminergic, noradrenergic, serotonergic, glutamatergic, and cholinergic systems within the brain. The hallmark finding of Parkinson's disease is degeneration of dopaminergic neurons within the substantia nigra pars compacta. The ventro-lateral tier, whose dopaminergic projections primarily connect to the dorsal putamen, is most severely affected in the early stage of Parkinson's disease.

The N-methyl-D-aspartate receptor (NMDAR) is critical for the facilitation of synaptic plasticity processes that drive learning and memory. Dopamine is a regulator of NMDAR function, and a dysregulation of the NMDAR activity is seen in Parkinson's disease, resulting in a loss of long-term potentiation and long-term depression and ultimately impairment of synaptic plasticity processes. As such, targeting the NMDAR may be an efficacious approach to treating cognitive impairment in Parkinson's disease.

A need continues to exist in the art for novel and more specific and/or potent compounds that are capable of treating cognitive impairment in Parkinson's disease.

### SUMMARY

In one aspect, provided herein are methods of treating cognitive impairment associated with neurodegenerative disease in a patient in need thereof, comprising administering daily to the patient an effective amount of a compound selected from (S) 2-(4-methoxybenzyl)-2,7-diazaspiro[3.5]nonane-1,6-dione (referred to herein as the “Compound,” or “Cpd”) and a pharmaceutically acceptable salt thereof. In some embodiments, the neurodegenerative disease is Parkinson's disease or Alzheimer's disease.

### BRIEF DESCRIPTION OF FIGURES

FIG. 1 depicts a timeline of events in an exploratory study of patients treated with the Compound.

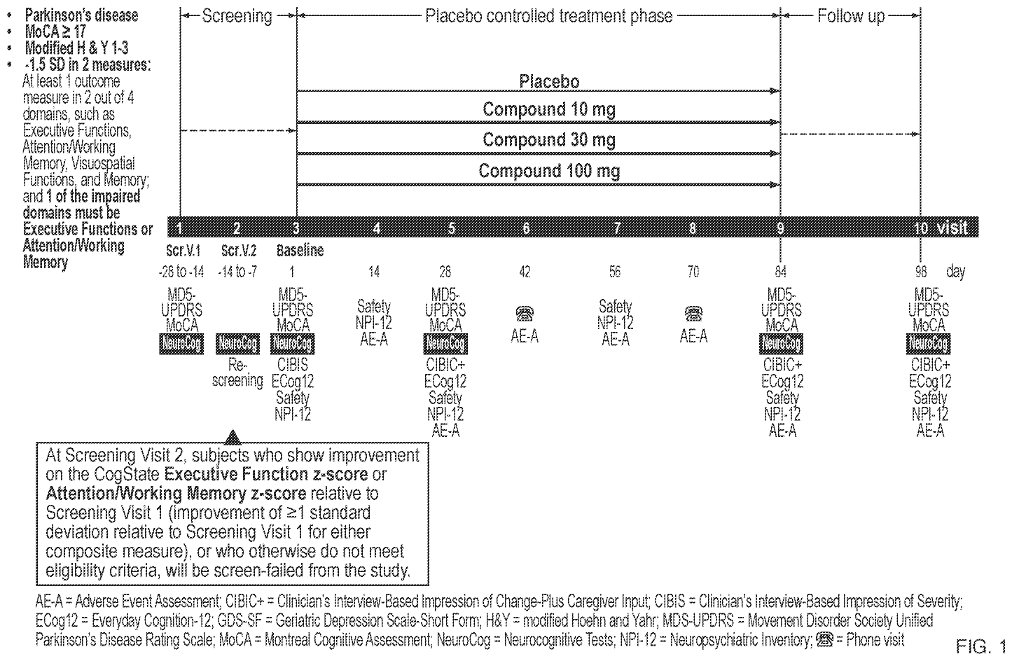

### DETAILED DESCRIPTION

As generally described herein, the present disclosure provides methods of treating cognitive impairment associated with neurodegenerative disease, using a compound of the disclosure, namely, (S) 2-(4-methoxybenzyl)-2,7-diazaspiro[3.5]nonane-1,6-dione (the “Compound”), or a pharmaceutically acceptable salt thereof.

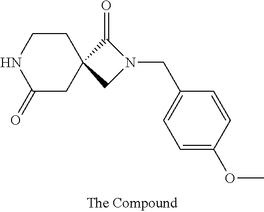 [SMILES: COc1ccc(CN2C[C@@]3(CCNC(=O)C3)C2=O)cc1]

The Compound is an orally available NMDAR modulator that binds to a unique site on the NMDAR, that acts as a glutamate coagonist with pharmacological properties distinct from known NMDAR agonists or antagonists. The Compound was well tolerated in both rat and dog, with a favorable pharmacokinetic profile. The Compound showed robust efficacy in rodent cognitive models (novel object recognition and Morris water maze). In the chronic low dose 1-methyl-4-phenyl-1,2,3,6-tetrahydropyridine (MPTP) non-human primate model of Parkinson's disease cognitive impairment, The Compound reversed MPTP-induced deficits in several cognitive domains, including attention, working memory, and executive function as measured through primate versions of Cambridge Neuropsychological Test Automated Battery assessments, with high translatability to clinical measures. The MPTP non-human primate study suggests a rapid, robust, and long lasting effect of the Compound.

### Definitions

To facilitate an understanding of the present invention, a number of terms and phrases are defined below.

### List of Abbreviations

| Abbreviation or | |
|---|---|
| Specialist Term | Explanation |
| APOE | Apolipoprotein E |
| CIBIC-Plus | Clinician's Interview-Based Impression of Change-Plus |
| | Caregiver Input |
| CIBIS | Clinician's Interview-Based Impression of Severity |
| COMT | Catechol orthomethyl-transferase |
| CW | Color-Word |
| DSST | Digit Symbol Substitution Test |
| ECog12 | Everyday Cognition, 12-item version |
| GDS-SF | Geriatric Depression Scale-Short Form |
| H&Y | Modified Hoehn and Yahr |
| MCI-PD | Mild cognitive impairment associated with Parkinson's |
| | Disease) |
| MDS-UPDRS | Movement Disorder Society Unified Parkinson's Disease |
| | Rating Scale |
| MMRM | Mixed model for repeated measures |
| MoCA | Montreal Cognitive Assessment |
| MPTP | 1-methyl-4-phenyl-1,2,3,6-tetrahydropyridine |
| NaSSA | noradrenergic and specific serotonergic antidepressant |
| NMDAR | N-methyl-D-aspartate receptor |
| NPI-12 | Neuropsychiatric Inventory |
| PDD | Parkinson's disease dementia |
| SA | Surface area (body) |
| SCOPA-Sleep | Scales for Outcomes in Parkinson's Disease-Sleep |
| | Disturbances |
| SNRI | serotonin and norepinephrine reuptake inhibitor |
| SSRI | selective serotonin reuptake inhibitor |
| S-STS | Sheehan Suicidality Tracking Scale |
| TEAE | treatment-emergent adverse event |
| USP-NF | United States Pharmacopeia and National Formulary |

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where formulations and kits are described as having, including, or comprising specific components, or where processes and methods are described as having, including, or comprising specific steps, it is contemplated that, additionally, there are formulation sand kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a formulation or a method described herein can be combined in a variety of ways without departing from the spirit and scope of the present invention, whether explicit or implicit herein. For example, where reference is made to a particular compound, that compound can be used in various embodiments of formulations of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, formulations specifying a percentage are by weight unless otherwise specified. Further, if a variable is not accompanied by a definition, then the previous definition of the variable controls.

As used herein, “pharmaceutical composition” or “pharmaceutical formulation” refers to the combination of a therapeutically active agent with a pharmaceutically acceptable excipient, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or a human, as appropriate, and/or that are approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that is present in a compound of the present invention (e.g., the Compound), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, K⁺, Ca²⁺, NH₄⁺, and NW₄⁺ (where W can be a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions or formulations of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, Pa. (1975).

The term “AUC” refers to the area under the time/plasma concentration curve after administration of the pharmaceutical formulation. AUC_{0-infnity} denotes the area under the plasma concentration versus time curve from time 0 to infinity; AUC₀₋ₜ denotes the area under the plasma concentration versus time curve from time 0 to time t. It should be appreciated that AUC values can be determined by known methods in the art.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

The term “Cₘₐₓ” refers to the maximum concentration of a therapeutic agent (e.g., the Compound) in the blood (e.g., plasma) following administration of the pharmaceutical formulation.

The term “tₘₐₓ” refers to the time in hours when Cₘₐₓ is achieved following administration of the pharmaceutical formulation comprising a therapeutic agent (e.g., the Compound).

As used herein, “solid dosage form” means a pharmaceutical dose(s) in solid form, e.g., tablets, capsules, granules, powders, sachets, reconstitutable powders, dry powder inhalers and chewables.

By “co-administer” it is meant that a formulation described herein is administered at the same time, just prior to, or just after the administration of one or more additional therapies (e.g., analgesic, anti-cancer agent, chemotherapeutic, or treatment for a neurodegenerative disease). The Compound, or a pharmaceutically acceptable salt thereof, can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”).

In general, an “effective amount” or “therapeutically effective amount” of a compound or a pharmaceutical formulation refers to an amount sufficient to elicit the desired biological response, e.g., to treat MCI-PD. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject.

As used herein, “PBO” is placebo.

As used herein, “Parkinson's Disease” diagnosis will be made per Movement Disorder Society criteria, with at least 1 year of Parkinson's disease symptoms.

As used here, “Cognitive Impairment” related to the MCI-PD diagnostic criteria will be defined in this protocol by:
≥1.5 standard deviations below age- and education-based norms in:
   At least 1 outcome measure in 2 out of 4 domains, such as Executive Functions, Attention/Working Memory, Visuospatial Functions, and Memory; and
   1 of the impaired domains should be Executive Functions or Attention/Working Memory.
Tools for diagnosis of cognitive impairment include:
   Executive Functions: Groton Maze Learning Test (from Cogstate), Two Back (from Cogstate), Stroop;
   Attention/Working Memory: Digit Symbol Substitution Test (DSST), One Back (from Cogstate), Identification Test (from Cogstate);
   Visuospatial Functions: Benson Complex Figure, Line Orientation;
   Memory: International Shopping List (from Cogstate), Continuous Paired Associate Learning (from Cogstate).

There must be no interference of cognitive impairment with function.

The frequency of MCI-PD was 42.5% using the Movement Disorder Society level 2 criteria at 1.5 standard deviations below normative values in the Incidence of Cognitive Impairment in Cohorts with Longitudinal Evaluation-Parkinson's Disease longitudinal observational study. The Movement Disorder Society MCI-PD criteria recommend at least 2 tests per domain with impairment demonstrated on at least 2 neuropsychological tests as either 2 impaired tests in 1 cognitive domain or 1 impaired test in 2 different cognitive domains. Using a 1.5 standard deviation cut-off yielded high sensitivity (93.8%) but lower specificity (60.7%).

As used herein, “CGI-S” is the clinical global impression scale. The CGI-Severity (CGI-S) asks the clinician one question: “Considering your total clinical experience with this particular population, how mentally ill is the patient at this time?” which is rated on the following seven-point scale: 1=normal, not at all ill; 2=borderline mentally ill; 3=mildly ill; 4=moderately ill; 5=markedly ill; 6=severely ill; 7=among the most extremely ill patients.

As used herein, “Hoehn and Yahr” scale is a commonly used system for describing how the symptoms of Parkinson's disease progress. To help describe the intermediate course of the disease, the following modified Hoehn and Yahr scale will be used:

Stage 1: Unilateral involvement only

Stage 1.5: Unilateral and axial involvement

Stage 2: Bilateral involvement without impairment of balance

Stage 2.5: Mild bilateral disease with recovery on pull test

Stage 3: Mild to moderate bilateral disease; some postural instability; physically independent

Stage 4: Severe disability; still able to walk or stand unassisted

Stage 5: Wheelchair bound or bedridden unless aided Subjects must have severity Stage 1 to 3, inclusive, on the modified Hoehn and Yahr scale.

As used herein, “Performance-Based Neurocognitive Assessments” refers to a set of neurocognitive tests, with their functions and domain-specificity summarized in the below Table 1.

For subjects on dopaminergic therapy (i.e., levodopa), all visits with cognitive assessments (Visits 1, 2, 3, 5, 6, and early termination) should be scheduled so that cognitive assessments are performed during the subjects' ON time. The Cogstate Screening tests may be repeated for eligibility determinations if the study participant encounters technical difficulties with the computer based testing platform. The investigative site should contact the medical monitor if the performance-based neurocognitive assessments need to be repeated more than one time.

TABLE 1
Neurocognitive Tests With Functions and Domain-Specificity

| | | Tests | | Basis of |
|---|---|---|---|---|
| | Domain | Used | Explor- | Exclusion |
| | Speci- | During | atory | at Visit |
| | ficity | Screening | Outcomes | 2⁽¹⁾ |
| Paper-and-Pencil | | | | |
| Stroop | EF | X | | |
| DSST | AWM | X | X | |
| Category Fluency | EF | | X | |
| Benson Complex | VS | X | X | |
| Figure Drawing | | | | |
| Line Orientation | VS | X | X | |
| Cogstate Tests | | | | |
| Groton Maze Learning | EF | X | X | X |
| Identification | AWM | X | X | X |
| Two Back | EF | X | X | X |
| One Back | AWM | X | X | X |
| International | ME | X | X | |
| Shopping List | | | | |
| Continuous Paired | ME | X | X | |
| Associate Learning | | | | |

AWM = attention/working memory;
DSST = Digit Symbol Substitution Test;
EF = executive functions;
ME = memory;
VS = visuospatial
⁽¹⁾Subjects who show improvement on the Cogstate Executive Function z-score (measured by composite score of Groton Maze Learning Test and Two Back Test) or Attention/Working Memory z-score (as measured by composite score of Identification Test and One Back) at Screening Visit 2 relative to Screening Visit 1 (improvement of ≥1 standard deviation relative to Screening Visit 1 for either composite measure) as measured by Cogstate will be withdrawn from the study.

As used herein, the “Stroop test” is a measure of executive functioning and processing speed. There are three components of the test: Word reading (W), Color naming (C), and the Color-Word (CW) condition. In Word reading, the subject is asked to read printed words (red, green, or blue) as quickly as they can. The W score is the number of correctly read words in 45 seconds. In Color naming, the stimulus page is filled with regularly spaced clusters of “XXXX,” where each cluster is printed in either red, green, or blue ink. The subject is asked to name the color of the ink of each cluster as quickly as they can. The C score is the number of correct responses within 45-seconds. In Color-Word condition, the stimulus page contains words that are the names of colors (red, green, or blue), but for each word, the color of the ink used to print the word is inconsistent with the word itself. Thus, the word “red” might be printed in green or blue ink. The subject is asked to state the color of the ink and not say the word. Thus, if the word was “red,” but it was printed in blue ink, the subject should respond “blue.” The score for CW is the number of correct responses in 45-seconds.

As used herein, “DSST” is Digit Symbol Substitution Test, and consists of digit-symbol pairs followed by a list of digits. Under each digit, the subject should write down the corresponding symbol as quickly as possible. The number of correct symbols within the allowed time is measured.

As used herein, “Category Fluency Test” is a test in which the subject is asked to name all different examples that can be recalled in 1 minute from a category. Rose, daisy, and marigold are among the correct examples for the category of flowers.

As used herein, “Benson Complex Figure Drawing test” is a test which assesses visuoconstructional and visual memory functions. The subject is presented with a figure composed of geometric shapes and asked to reproduce the figure on the same page. The accuracy of each shape and its placement is recorded. Each figure element is scored as follows:
2 points if the element is drawn accurately and placed correctly in the figure (1 point for accuracy and 1 point for placement.
1 point if the element is poorly drawn but placed correctly or is correctly drawn but misplaced.
0 points if the element is neither accurately drawn nor correctly placed.

As used herein, the “Line Orientation Test” is a standardized test of visuospatial skills commonly associated with functioning of the parietal lobe in the right hemisphere. The test measures a person's ability to match the angle and orientation of lines in space.

As used herein, “Cogstate test” is a test from Cogstate, a cognitive science company focused on the measurement of cognition, and provides rapid, reliable, sensitive, and simple computerized cognitive tests for clinical trials, academic research, healthcare, and brain injury. Individual tests comprising the customized battery of cognitive tests used for this protocol are described as follows. The Cogstate tests include the Groton Maze Learning test, the Identification test, the Two Back test, the One Back test, the International Shopping List test, and the Continuous Paired Associate Learning test.

As used herein, “Groton Maze Learning test” is a measure of problem solving and reasoning and uses a well-validated maze learning paradigm. In this test, the subject is shown a 10×10 grid of boxes on a computer screen. A 28-step pathway is hidden among these 100 possible locations. Each box represents move locations, and the grid refers to the box array (i.e., 10×10). Subjects are required to find the hidden pathway guided by 3 search rules. These rules are: do not move diagonally, do not move more than 1 box (i.e., do not jump), and do not move back on the pathway. At each step, only the most recently selected box is shown. Subjects do not need to return to the last correct location after making an error before continuing their search for the next correct box. Feedback is given with visual and auditory cues (green check marks and red crosses) to indicate whether the selected box is correct or incorrect. All correctly selected boxes remain ticked with a green check, keeping the revealed pathway displayed until the round is completed. There are 20 well-matched alternate pathways available. The software records each move as an error or as a correct move.

As used herein, the “Identification test” is a measure of visual attention and uses a well-validated choice reaction time paradigm with playing-card stimuli. In this test, the playing cards are all either red or black jokers. The subject is asked whether the card displayed in the center of the screen is red. The subject responds by pressing the Yes key when the joker card is red and No when it is black. The software measures the speed and accuracy of each response.

As used herein, the “Two Back test” is a measure of working memory and uses a well-validated, n-back paradigm with playing-card stimuli. In this test, the playing cards are identical to those found in a standard deck of 52 playing cards (without the joker cards). The subject is asked whether the card displayed in the center of the screen is the same as the one presented 2 cards previously. The subject responds by pressing the Yes or No key. Because no card has been presented 2-back on the first and second trials, a correct response on these trials is always No.

As used herein, the “One Back test” is a measure of working memory and uses a well-validated, n-back paradigm with playing-card stimuli. In this test, the playing cards are identical to those found in a standard deck of 52 playing cards (without the joker cards). The subject is asked whether the card displayed in the center of the screen is the same as the card presented immediately previously. The subject responds by pressing the Yes or No key. Because no card has been presented yet on the first trial, a correct first response is always No. The software measures the speed and accuracy of each response.

As used herein, the “International Shopping List test” is a measure of verbal learning and uses a well-validated list-learning paradigm. The test is administered using a computer. High frequency, high imagery, concrete nouns (items from a shopping list) are read to the subject by the test supervisor at the rate of 1 word every 2 seconds. Once all (12) words have been read, the subject is asked to recall as many of the words as he/she can as quickly as possible. The test supervisor uses a mouse or stylus to mark the words recalled by the subject on the computer screen. When the subject can recall no more words, the same list is read a second time. The test supervisor records the words recalled by the subject on this trial. This is then repeated a third time. The software measures the number of correct responses, as recorded by the test supervisor.

As used herein, the “Continuous Paired Associate Learning test” is a measure of visual associate memory and uses a well-validated, paired associate learning paradigm in which the subject must learn the locations of a number of amoeba-like shapes on the computer screen. This test consists of a single amoeboid shape displayed in the center of the screen surrounded by a number of blue-filled circles. In the exposure phase of the test all the to-be-remembered pattern-location associations are presented on the computer screen simultaneously. After a 5-second delay, a pattern is shown in the central location and this signals that the subject should touch the location in the periphery that contains the same pattern. This process continues until the participant has acknowledged all the pattern-location associations. The learning phase begins with the same test display presented during the exposure phase except that now all of the peripheral locations are shown as blue spheres. One of the patterns presented in the exposure phase is presented in the center location. With the presentation of this pattern, the subject is required to select the peripheral location where an identical pattern is hidden beneath the blue sphere. This process continues until the correct location of each pattern is found. Finding the correct location for all patterns in the set is defined as a learning trial. There are 6 learning trials. The software records each move as an error or as a correct move.

As used herein, “PDAQ-15” is the 15-item Penn Parkinson's Disease Activities Questionnaire.

### Pharmaceutical Formulations

In one aspect, provided herein is a pharmaceutical formulation of (S) 2-(4-methoxybenzyl)-2,7-diazaspiro[3.5]nonane-1,6-dione (the “Compound”), or a pharmaceutically acceptable salt thereof, and a pharmaceutically acceptable excipient, which can be used for the treatment of cognitive impairment associated with neurodegenerative disease.

In certain embodiments, the pharmaceutical formulation comprises the Compound or a pharmaceutically acceptable salt thereof; and a pharmaceutically acceptable excipient.

In some embodiments, the pharmaceutical formulation comprises the Compound, or a pharmaceutically acceptable salt thereof, present in a therapeutically effective amount; and a pharmaceutically acceptable excipient. In certain embodiments, the pharmaceutical formulation comprises an effective amount of the Compound. In certain embodiments, the pharmaceutical formulation comprises an effective amount of a pharmaceutically acceptable salt of the Compound. In some embodiments, the pharmaceutically acceptable excipient is one or more of microcrystalline cellulose; pregelatinized starch, and magnesium stearate.

In certain embodiments, the amount of the Compound, or a pharmaceutically acceptable salt thereof, in the pharmaceutical formulation is about 10 mg, about 30 mg, or about 100 mg. In some embodiments, the amount of the Compound, or a pharmaceutically acceptable salt thereof, in the pharmaceutical formulation is about 10 mg. In some embodiments, the amount of the Compound, or a pharmaceutically acceptable salt thereof, in the pharmaceutical formulation is about 30 mg. In some embodiments, the amount of the Compound, or a pharmaceutically acceptable salt thereof, in the pharmaceutical formulation is about 100 mg.

In some embodiments, the pharmaceutical formulation is in a solid dosage form encapsulated in a capsule. In some embodiments, the capsule comprises hydroxyl-propyl cellulose. In some embodiments, the capsule comprises about 10 mg of the Compound, or a pharmaceutically acceptable salt thereof; about 30 mg of the Compound, or a pharmaceutically acceptable salt thereof; or about 100 mg of the Compound, or a pharmaceutically acceptable salt thereof.

In some embodiments, the pharmaceutical formulation is in a solid dosage form in the form of a tablet. In some embodiments, the tablet comprises about 10 mg of the Compound, or a pharmaceutically acceptable salt thereof; about 30 mg of the Compound, or a pharmaceutically acceptable salt thereof; or about 100 mg the Compound, or a pharmaceutically acceptable salt thereof.

In some embodiments, about 10 mg of the Compound or a pharmaceutically acceptable salt thereof is provided as a unit dose. In some embodiments, about 30 mg of the Compound or a pharmaceutically acceptable salt thereof is provided as a unit dose. In some embodiments, about 100 mg of the Compound or a pharmaceutically acceptable salt thereof is provided as a unit dose.

In certain embodiments, the pharmaceutical formulation is a formulation for oral administration.

Although the descriptions of pharmaceutical formulations provided herein are principally directed to pharmaceutical formulations which are suitable for administration to humans, it will be understood by the skilled artisan that such formulations are generally suitable for administration to animals of all sorts. Modification of pharmaceutical formulations suitable for administration to humans in order to render the formulations suitable for administration to various animals is well understood, and the ordinarily skilled veterinary pharmacologist can design and/or perform such modification with ordinary experimentation. General considerations in the formulation and/or manufacture of pharmaceutical formulations can be found, for example, in Remington: The Science and Practice of Pharmacy 21^{st} ed., Lippincott Williams & Wilkins, 2005.

### Methods of Use and Treatment

In one aspect, provided herein are methods of treating cognitive impairment associated with neurodegenerative disease in a patient in need thereof, comprising administering daily to the patient an effective amount of a compound selected from (S) 2-(4-methoxybenzyl)-2,7-diazaspiro[3.5]nonane-1,6-dione and a pharmaceutically acceptable salt thereof.

In some embodiments, the neurodegenerative disease is Parkinson's disease or Alzheimer's disease.

In some embodiments, the neurodegenerative disease is Parkinson's disease.

In some embodiments, the cognitive impairment is mild cognitive impairment.

In some embodiments, the method comprises orally administering about 10 mg, about 30 mg or about 100 mg of the compound daily. In some embodiments, the the method comprises orally administering about 30 mg of the compound daily.

In some embodiments, the patient has a Montreal Cognitive Assessment Score before administration of 17 or more. In some embodiments, the patient have a Montreal Cognitive Assessment Score before administration of between 15 to 25.

In some embodiments, the patient has a score of at least 3 on the Clinical Global Impression Scale (CGI-S) for mental illness.

In some embodiments, the patient has cognitive impairment in at least one of the following cognitive domains: attention, mental processing speed, executive functioning/problem solving, and visuospatial function.

In some embodiments, the patient has cognitive impairment as defined by scoring below or about 1.5 standard deviations of age and education based norms in a least one of executive function or attention/working memory cognitive domains, using one or more of: Movement Disorder Society MCI-PD diagnostic criteria, Groton Maze Learning Test, Digit Symbol Substitution Test (DSST), Cogstate One Back test, Cogstate Identification Test or the Stroop test, before administration of the compound.

In some embodiments, the patient has dementia related psychosis.

In another aspect, provided herein are methods of treating cognitive impairment associated with Parkinson's disease in a patient having a Montreal Cognitive Assessment of 17 or more, comprising administering daily to the patient an effective amount of a compound selected from (S) 2-(4-methoxybenzyl)-2,7-diazaspiro[3.5]nonane-1,6-dione and a pharmaceutically acceptable salt thereof.

In some embodiments, the patient is currently being administered levodopa and/or a levodopa enhancer.

In some embodiments, after 12 weeks or more of daily administration of the compound, the patient has improved or stabilized cognition as measured by one or more of: improvement in a composite z-score based on DSST, Category Fluency Test Two Back Test, One Back Tests, Identification Test, and Groton Maze Learning Tests of Executive Function and Attention/Working Memory.

In some embodiments, after daily administration of the compound for 12 weeks or more, the patient shows improvement in an Executive Functions composite score as assessed by one or more of Groton Maze Learning Test, Two Back Test and Category Fluency Test, as compared to baseline.

In some embodiments, after daily administration of the compound for 12 weeks or more, the patient has improvement in an Attention/Working Memory composite score as assessed by one or more of DSST, One Back Test, and Identification Test.

In some embodiments, after daily administration of the compound for 12 weeks or more, the patient has memory improvement as measured by a Memory composite score using an International Shopping List or Continuous Paired Associate tests.

In some embodiments, after daily administration of the compound for 12 weeks or more, the patient has improved sleep quality as measured by the Scales for Outcomes in Parkinson's Disease Sleep Disturbances (SCOPA Sleep) compared to baseline.

In some embodiments, after 12 weeks or more of daily administration of the compound, the patient has the same or better cognition as measured by the MoCA.

In some embodiments, after 12 weeks or more of daily administration of the compound, the patient has improvement in daily functions as measured by the 12-item version of Everyday Cognition (ECog12).

In some embodiments, after 12 weeks or more of daily administration of the compound, the patient has improvement as measured by the 12-item version of Penn Parkinson's Disease Activities Questionnaire (PDAQ-15)).

In some embodiments, the compound is administered as part of a capsule or a tablet.

In some embodiments, administering comprises administering daily to the patient a pharmaceutical formulation, wherein the pharmaceutical formulation comprises the Compound, or a pharmaceutically acceptable salt thereof, present in a therapeutically effective amount; microcrystalline cellulose; pregelatinized starch, and magnesium stearate.

In some embodiments, the pharmaceutical formulation is encapsulated in a capsule. In some embodiments, the capsule comprises hydroxyl-propyl cellulose. In some embodiments, the capsule comprises about 10 mg of the Compound, or a pharmaceutically acceptable salt thereof; about 30 mg of the Compound, or a pharmaceutically acceptable salt thereof; or about 100 mg the Compound, or a pharmaceutically acceptable salt thereof. In some embodiments, the capsule comprises about 30 mg of the Compound, or a pharmaceutically acceptable salt thereof.

In some embodiments, the pharmaceutical formulation is in the form of a tablet. In some embodiments, the tablet comprises about 10 mg of the Compound, or a pharmaceutically acceptable salt thereof; about 30 mg of the Compound, or a pharmaceutically acceptable salt thereof; or about 100 mg of the Compound, or a pharmaceutically acceptable salt thereof. In some embodiments, the tablet comprises about about 30 mg of the Compound, or a pharmaceutically acceptable salt thereof.

In some embodiments, the patient is human.

The pharmaceutical methods provided herein can include administration by oral (enteral) administration. That is, in some embodiments, the methods include administering orally a compound or a pharmaceutical formulation disclosed herein.

The methods provided herein may also include chronic administration. Chronic administration refers to administration of a compound or pharmaceutical formulation comprising a compound disclosed herein over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

The pharmaceutical formulations provided herein may be presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions.

In certain embodiments, the pharmaceutical formulations provided herein are administered to the patient as a solid dosage form. In some embodiments, the solid dosage form is a capsule.

In certain embodiments, the compounds provided herein can be administered as the sole therapeutically active agent, or they can be administered in combination with other therapeutically active agents.

In certain embodiments, administering an effective amount of the Compound, or a pharmaceutically acceptable salt thereof, comprises administering orally about 10 mg of the Compound, or a pharmaceutically acceptable salt thereof, once daily.

In certain embodiments, administering an effective amount of the Compound, or a pharmaceutically acceptable salt thereof, comprises administering orally about 30 mg of the Compound, or a pharmaceutically acceptable salt thereof, once daily.

In certain embodiments, administering an effective amount of the Compound, or a pharmaceutically acceptable salt thereof, comprises administering orally about 100 mg of the Compound, or a pharmaceutically acceptable salt thereof, once daily.

In some embodiments, the about 10 mg of the Compound or a pharmaceutically acceptable salt thereof is provided as a unit dose.

In some embodiments, the about 30 mg of the Compound or a pharmaceutically acceptable salt thereof is provided as a unit dose.

In some embodiments, the about 100 mg of the Compound or a pharmaceutically acceptable salt thereof is provided as a unit dose.

Also provided herein are combination therapies comprising a compound of the disclosure, e.g., the Compound, in combination with one or more other active agents. For example, a compound may be combined with one or more anti-Parkinson's medications, such as levadopa, carbidopa, safinamide, amantadine, trihexyphenidyl, benztropine, selegiline, rasagiline, rivastigmine, or dopamine agonists (e.g., ropinirole, pramipexole, or rotigotine). A compound may also be combined with one or more analgesics, such as non-steroidal anti-inflammatory agents (NSAIDS), steroidal anti-inflammatory agents, opiates, and cyclo-oxygenase inhibitors. A compound may also be combined with other agents such as antidepressants, such as tricyclic antidepressants, MAO-I's, SSRI's, SNRI's, and double and triple uptake inhibitors and/or anxiolytic drugs. Exemplary drugs that may be used in combination with a compound include Anafranil, Adapin, Aventyl, Elavil, Norpramin, Pamelor, Pertofrane, Sinequan, Surmontil, Tofranil, Vivactil, Parnate, Nardil, Marplan, Celexa, Lexapro, Luvox, Paxil, Prozac, Zoloft, Wellbutrin, Effexor, Remeron, Cymbalta, Desyrel (trazodone), and Ludiomill. In another example, a compound may be combined with an antipsychotic medication. Non-limiting examples of antipsychotics include butyrophenones, phenothiazines, thioxanthenes, clozapine, olanzapine, risperidone, quetiapine, ziprasidone, amisulpride, asenapine, paliperidone, iloperidone, zotepine, sertindole, lurasidone, and aripiprazole. In another example, a compound, e.g., the Compound, may be combined with an antiepileptic medication. Non-limiting examples of antiepileptics include gabapentin, pregabalin, carbamazepine, clonazepam, lacosamide, lamotrigine, levetiracetam, oxcarbazepine, phenytoin, pregabalin, topiramate, and valproate. Use of any of the drugs named herein can include use of its branded or generic equivalents. It should be understood that combinations of a compound, e.g., the Compound, or a pharmaceutically acceptable salt thereof, and one or more of the above therapeutics may be used for treatment of any suitable condition and are not limited to use as anti-pain medication.

### Examples

In order that the disclosure described herein may be more fully understood, the following examples are set forth. The synthetic and biological examples described in this application are offered to illustrate the compounds, pharmaceutical formulations, and methods provided herein and are not to be construed in any way as limiting their scope.

The compounds described herein can be prepared in a number of ways based on the teachings contained herein and synthetic procedures known in the art. The following non-limiting examples illustrate the disclosure.

All sample preparation and measurement was done in ambient air atmosphere at about one atmosphere of pressure.

### Chemical Definitions

Herein, the following chemical definitions are used. “DCC” is dicyclohexylcarbodiimide, “DCM” is dichloromethane, “DCU is dicyclohexvlurea, “¹H-NMR” is proton nuclear magnetic resonance. “LCMS” is liquid chromatography-mass spectrometry, “LiHMDS” is lithium bis(trimethylsilyl)amide, “PMB” is para-methoxybenzyl, “RT” is room temperature, “TEA” is triethylamine, “THF” is tetrahydrofuran, and “TLC” is thin-layer chromatography.

### Example 1: Synthesis of The Compound

### Synthesis of AA-rac, AA-1 & The Compound

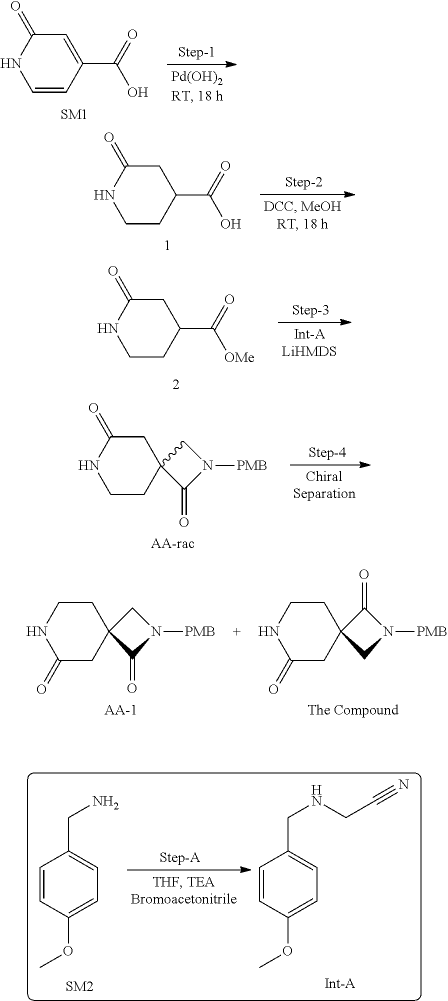

### Synthesis of 2-oxopiperidine-4-carboxylic acid (1)

To a stirred solution of 2-oxo-1,2-dihydropyridine-4-carboxylic acid (SM1) (500 g, 3.59 mol) in methanol (10 L) was added palladium hydroxide (150 g) into a 20 L autoclave under N₂ atmosphere. The reaction mixture was stirred under H₂ atmosphere (5 kg/cm²) at RT for 18 h. After consumption of the starting material (monitored by LCMS), the reaction mixture was filtered through a pad of celite and concentrated under reduced pressure. Obtained reside was triturated with diethyl ether (2.5 L) and dried under vacuum to afford compound 1 (450 g, 85%) as off white solid. ¹H NMR (500 MHz, DMSO-d₆): δ 12.37 (s, 1H), 7.44 (s, 1H), 3.16-3.12 (m, 2H), 2.78-2.71 (m, 1H), 2.33-2.21 (m, 2H), 1.98-1.92 (m, 1H), 1.74-1.63 (m, 1H)

LCMS (m/z): 144.2 [M⁺+1].

### Synthesis of methyl 2-oxopiperidine-4-carboxylate (2)

To a stirring solution of compound 1 (250 g, 1.74 mol) in MeOH (2.5 L) was added DCC (540 g, 2.62 mol) under nitrogen atmosphere at RT and stirred for 16 h. After consumption of the starting material (by TLC), reaction mixture was diluted with diethyl ether (2.5 L) and allowed to stir for 2 h. The obtained solid (DCU) was filtered off, the filtrate was concentrated under reduced pressure to obtain a solid having desired compound along with some amounts of DCU and other impurities. This solid after another cycle of trituration with Et₂O (2 L) was treated with MeOH:EtOAc (1:9, 3 L) for 3 h. The undissolved solid (DCU) was filtered off and the filtrate was evaporated to afford the desired compound (100 g, 36%) as an off white solid. ¹H NMR (400 MHz, DMSO-d₆) δ 7.53 (s, 1H), 3.63 (s, 3H), 3.18-3.09 (m, 2H), 2.94-2.82 (m, 1H), 2.86-2.74 (m, 2H), 2.04-1.97 (m, 1H), 1.79-1.69 (m, 1H). LCMS (m/z): 158.2 [M⁺+1].

### Synthesis of 2-(4-methoxybenzyl)-2,7-diazaspiro[3.5]nonane-1,6-dione (AA-rac, AA-1 & The Compound)

To a stirred solution of compound 2 (150 g, 0.955 mol) in dry THF (1.25 L) was added LiHMDS (1.0 M in THF, 1.9 L, 1.91 mol) slowly at −78° C. under nitrogen atmosphere. The reaction temperature was raised to −20° C. and stirred for 1 h. Int-A (167 g, 0.955 mol) in THF (250 mL) was added drop wise at −78° C. The reaction mixture was brought to room temperature and stirred for 16 h. After consumption of the starting material (by TLC), the reaction was quenched with aqueous NH₄Cl (1 L) at 0° C., added Et₂O (1.5 L) and stirred for 10 minutes. Organic layer was separated and aqueous layer was extracted with 10% MeOH/CH₂ Cl₂. The combined organic layer was washed with brine, dried over Na₂SO₄ and concentrated to obtain racemic AA-rac (110 g, 40%) as an off white solid. Two more batches were repeated and obtained 125 g racemic AA-rac. All batches were combined to obtain racemic AA-rac (230 g) as an off white solid. AA-rac (200 g) was purified by chiral HPLC purification to obtain AA-1 (94 g) as a white solid and the Compound (92 g) as a white solid.

AA-1: ¹H NMR (500 MHz, DMSO-d₆) δ 7.56 (br s, 1H), 7.17 (d, J=8.7 Hz, 2H), 6.93 (d, J=8.7 Hz, 2H), 4.32-4.18 (m, 2H), 3.74 (s, 3H), 3.35-3.25 (m, 1H), 3.19-3.10 (m, 1H), 3.07 (d, J=5.2 Hz, 1H), 2.98 (d, J=5.8 Hz, 1H), 2.36 (s, 2H), 1.94-1.80 (m, 2H). LCMS (ESI): m/z 275.2 [M⁺+1]. HPLC: 99.88%. Chiral HPLC: 100.00%. Column: CHIRALPAK IC (250*4.6 mm, 3.5 m); Mobile Phase A: 0.1% DEA in n-hexane; Mobile Phase B: DCM:MeOH (50:50); A:B:: 60:40; Flow rate: 1.0 mL/min; Retention time: 13.840.

The Compound: ¹H NMR (500 MHz, DMSO-d₆) δ 7.56 (br s, 1H), 7.17 (d, J=8.1 Hz, 2H), 6.93 (d, J=8.7 Hz, 2H), 4.31-4.19 (m, 2H), 3.74 (s, 3H), 3.35-3.26 (m, 1H), 3.18-3.10 (m, 1H), 3.07 (d, J=5.2 Hz, 1H), 2.98 (d, J=5.8 Hz, 1H), 2.36 (s, 2H), 1.96-1.80 (m, 2H). LCMS (ESI): m/z 275.2 [M⁺+1]. HPLC: 99.42%. Chiral HPLC: 100.00%. Column: CHIRALPAK IC (250*4.6 mm, 3.5 μm); Mobile Phase A: 0.1% DEA in n-hexane; Mobile Phase B: DCM:MeOH (50:50); A:B:: 60:40; Flow rate: 1.0 mL/min; Retention time: 15.543.

### Preparation of Int-A: Synthesis of 2-((4-methoxybenzyl)amino)acetonitrile (Int-A)

To a solution of 4-methoxy benzyl amine (750 g, 5.47 mol) in THF (4.5 L) was added TEA (1.1 Kg, 10.9 mol) and 2-bromoacetonitrile (788 g, 6.59 mol) at 0° C. and stirred for 16 h under nitrogen atmosphere. After consumption of the starting material (by TLC), the reaction was quenched with water (2 L) and extracted with EtOAc (2×1.5 L). The combined organic layer was dried over Na₂SO₄ and concentrated under reduced pressure to obtain crude compound which was purified by column chromatography by eluting with 40% EtOAc/n-hexane to afford Int-A (675 g, 70% yield with 96% purity by HPLC) as a liquid. ¹H NMR (500 MHz, DMSO-d₆) δ 7.21 (d, J=9.0 Hz, 2H), 6.86 (d, J=9.0 Hz, 2H), 3.78 (s, 3H), 3.72 (s, 2H), 3.56 (s, 2H), 2.93 (br s, 2H). LCMS (m/z): 177.1 [M⁺+1].

### X-Ray Crystal Structure Determination of The Compound

The Compound (40 mg) was dissolved in methanol (2 mL) at 40° C., allowed to cool to room temperature and was left standing for 72 h to form crystals. Crystals were isolated and examined with a microscope. Single crystal X-ray diffraction analysis also was performed, which shows that the crystals are orthorhombic and have a P2₁2₁2₁ space group. Analysis of the single crystal diffraction data shows that the absolute configuration of the carbon at the spiro center is (S), as determined by the PLATON technique (A. L. Spek, J. Appl. Cryst., 36, 7-13 (2003)). Based on these results, the absolute stereochemistry of compound the Compound is shown in the structure below:

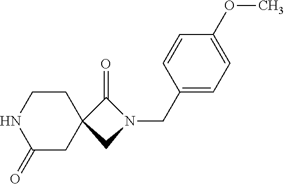

### Example 2: A Study to Evaluate the Compound in Subjects with Mild Cognitive Impairment Associated with Parkinson's Disease

### Objectives and Endpoints:

The objective of the study is to evaluate the safety and tolerability of multiple dose levels of the Compound in subjects with mild cognitive impairment associated with Parkinson's disease (MCI-PD), and to explore the potential benefit of the Compound on cognition.

### Primary Objective:

To evaluate the safety and tolerability of multiple dose levels of the Compound in subjects with MCI-PD.

### Exploratory Objectives:

To explore the potential cognitive and functional effects of multiple dose levels of the Compound.
To explore the effect of multiple dose levels of the Compound on non-motor symptoms associated with Parkinson's disease.
To explore the global clinical effect of the Compound as measured by Clinician's Interview-Based Impression of Change-Plus Caregiver Input (CIBIC-Plus).
To explore the effect of multiple dose levels of the Compound on motor symptoms associated with Parkinson's disease.
To evaluate the plasma concentrations of the Compound.
To explore symptom profile and response to the Compound intervention as function of catechol orthomethyl-transferase (COMT) polymorphism (Met/Met, Met/Val, and Val/Val) and apolipoprotein E (APOE) polymorphisms.
To explore potential relationships between pharmacogenomics and efficacy.

The neurocognitive tests for this study, with their functions and domain-specificity, are summarized below in Table 2.

TABLE 2
Neurocognitive Tests With Functions and Domain-Specificity

| | | Tests | | Basis of |
|---|---|---|---|---|
| | Domain | Used | Explor- | Exclusion |
| | Speci- | During | atory | at Visit |
| | ficity | Screening | Outcomes | 2^{a} |
| Paper-and-Pencil | | | | |
| Stroop | EF | X | | |
| DSST | AWM | X | X | |
| Category Fluency | EF | | X | |
| Benson Complex | VS | X | X | |
| Figure Drawing | | | | |
| Line Orientation | VS | X | X | |
| Cogstate Tests | | | | |
| Groton Maze Learning | EF | X | X | X |
| Identification | AWM | X | X | X |
| Two Back | EF | X | X | X |
| One Back | AWM | X | X | X |
| International | ME | X | X | |
| Shopping List | | | | |
| Continuous Paired | ME | X | X | |
| Associate Learning | | | | |

AWM = attention/working memory;
DSST = Digit Symbol Substitution Test;
EF = executive functions;
ME = memory;
VS = visuospatial
^{a}Subjects who show improvement on the Cogstate Executive Function z-score (measured by composite score of Groton Maze Learning Test and Two Back Test) or Attention/Working Memory z-score (as measured by composite score of Identification Test and One Back) at Screening Visit 2 relative to Screening Visit 1 (improvement of ≥1 standard deviation relative to Screening Visit 1 for either composite measure) as measured by Cogstate will be withdrawn from the study.

### Methodology:

This study will include a 14- to 28-day Screening Period; a 12-week (84-day) double-blind, randomized, placebo-controlled Treatment Period; and a 14-day Follow-Up Period.

### Screening Period (Day −28 to Day −1)

At Screening Visit 1, potential subjects and study partners will be asked to provide written informed consent before completing any protocol specified procedures. Subjects with a diagnosis of Parkinson's disease based on Movement Disorder Society diagnostic criteria, with severity stage of 1 to 3 on the modified Hoehn and Yahr scale, score of ≥17 on the Montreal Cognitive Assessment (MoCA), and score of ≤8 points on the Geriatric Depression Scale-Short Form (GDS-SF) will be screened to determine initial eligibility. A diagnostic battery of paper-based and computer-based neuropsychological and cognitive tests, along with clinical measures and safety assessments, will be completed. Screening Visits 1 and 2 should be scheduled at approximately the same time of day. For subjects on dopaminergic therapy (i.e., levodopa), all visits with cognitive assessments (Visits 1, 2, 3, 5, 9, 10, and early termination) should be scheduled so that cognitive assessments are performed during the subjects' ON time.

Screening Visits 1 and 2 should be separated by ≥7 days. At Screening Visit 2, neuropsychological and cognitive testing will be completed. Subjects who show improvement on the Cogstate Executive Function z-score (measured by composite score of Groton Maze Learning Test and Two Back Test) or Attention/Working Memory z-score (as measured by composite score of Identification Test and One Back) relative to Screening Visit 1 (improvement of ≥1 standard deviation relative to Screening Visit 1 for either composite measure) as assessed by Cogstate, or who otherwise do not meet eligibility criteria, will be screen-failed from the study. Subjects who do not meet eligibility criteria according to an Enrollment Authorization Committee (EAC) will be screen-failed from the study.

Total duration of the Screening Period will be 14 to 28 days, depending on scheduling needs. The Screening Period may be extended if necessary, and an extension should be discussed with the sponsor-designated Medical Monitor.

### 12-Week Treatment Period:

### Baseline Visit (Visit 3, Day 1)

Screening Visit 2 and Baseline Visit (Visit 3) should be separated by ≥7 days. Cognitive function, motor and non-motor symptoms of Parkinson's disease, neuropsychological function, sleep, and global severity will be assessed. Subjects who meet eligibility criteria will be randomized to receive 1 of 3 doses of the Compound (10, 30, and 100 mg), or matching placebo once daily without food. Subjects should not consume food for 2 hours before each dose or 1 hour after each dose of study drug. The first dose of study drug will be administered at the clinic after the neurocognitive tests are administered. Study drug will be dispensed for subsequent outpatient administration.

### Visits 4 to 9 (Days 14, 28, 42, 56, and 70, and 84)

Subjects will take the Compound or matching placebo once daily for 12 weeks without food and will return to the clinic for evaluation on Days 14, 28, 56 and 84. Abbreviated clinical safety visits will be completed on Days 14 and 56. Cognitive function, motor and non-motor symptoms of Parkinson's disease, neuropsychological function, sleep, and global change will be assessed on Days 28 and 84 (end of treatment period). Phone visits will be completed on Days 42 and 70 to assess adverse events and concomitant medication changes.

Follow-Up Period (Day 98 or 14 days post-therapy)

Subjects will return for a follow-up visit on Day 98, or approximately 14 days following the final dose of study drug. Cognitive function, motor and non-motor symptoms of Parkinson's disease, neuropsychological function, sleep, and global change will be assessed. Procedures and assessments for subjects who prematurely discontinue are identified in the schedule of assessments table.

### Other Assessments and Information

Safety will be assessed throughout the entire study by adverse events, concomitant medications, vital signs, body weight, electrocardiograms, clinical laboratory values, and physical examinations as indicated in the schedule of assessments table. Additional blood samples will be collected for COMT and APOE pharmacogenetics/polymorphism testing, plasma concentration measurement, and pharmacogenomics DNA (optional) testing. Rater qualification, training, and surveillance will be centrally managed during the study.

### Number of Subjects:

Approximately 135 randomized subjects.

### Diagnosis and Main Criteria for Eligibility:

### Inclusion Criteria:

1. An Institutional Review Board-approved written informed consent and privacy language (Health Insurance Portability and Accountability Act) authorization obtained from the subject and study partner prior to performing any study-related procedures.
2. Male and female subjects from 50 to 80 years of age, inclusive.
3. Diagnosis of Parkinson's disease per Movement Disorder Society criteria, with at least 1 year of Parkinson's disease symptoms.
4. Diagnosis of mild cognitive impairment associated with Parkinson's Disease.
5. MoCA score ≥17.
6. Objective cognitive impairment as defined in this protocol by ≥1.5 standard deviations below age- and education-based norms in:
   At least 1 outcome measure in 2 out of 4 domains, such as Executive Functions, Attention/Working Memory, Visuospatial Functions, and Memory; and
   1 of the impaired domains must be Executive Functions or Attention/Working Memory.
7. Modified Hoehn and Yahr Stage 1 to 3, inclusive, and as assessed during the subject's ON time status.
8. Stable anti-parkinsonian regimen with dopamine agonists, or levodopa/levodopa enhancer for at least 6 weeks prior to Screening Visit 1 and expected to continue at the same dose during the study. NOTE: No dose adjustments within 6 weeks prior to screening or during the study.
9. Male subjects who are sexually active with female partner(s) must agree to the following during the study and for 30 days after the last dose of study drug: a) use an acceptable method of birth control (condom with spermicide or surgical sterilization) and b) refrain from sexual activity with female partners who do not use an acceptable method of birth control. Barrier contraception (condom with spermicide) must be used by all male subjects who are not surgically sterilized at least 90 days prior to screening. Male subjects must also agree to refrain from sperm donation during the study and until 90 days after the last dose of study drug.
10. Female subjects of childbearing potential must have a negative serum pregnancy test at screening and be practicing an adequate method of birth control (e.g., oral or parenteral contraceptives, intrauterine device, barrier, abstinence). Subjects may not be breastfeeding or plan to become pregnant or donate ova during the study and for 30 days after the last dose of study drug.
11. Minimum of 6th grade education or equivalent.
12. Sufficient visual acuity and motor control to complete assessments in the Investigator's opinion.
13. Ability to complete tests administered with computers or electronic tablets (as assessed by Cogstate).
14. Has a study partner who, in the Investigator's judgment, is able to provide information on cognitive, behavioral, and functional status of the subject for as long the subject is in the study. In the Investigator's judgment, study partner has frequent and sufficient contact (e.g., 5 times/week, or approximately 10 hours/week) with the subject. Study partner must accompany the subject at Visit 1 and Visit 3.
15. Ability to understand the requirements of the study, abide by the study restrictions, and agree to return for the required assessments.
16. Approved as an appropriate and suitable study participant by the EAC.

### Exclusion Criteria:

1. Clinically meaningful motor complications [e.g. reduced duration of levodopa antiparkinsonian action (wearing off phenomenon), sudden shifts between under-treated and over-treated states (on-off phenomenon), freezing and involuntary movements such as levodopa-induced dyskinesia].
2. Improvement on the Cogstate Executive Function z-score (measured by composite score of Groton Maze Learning Test and Two Back) or Attention/Working Memory z-score (measured by composite score of Identification Test and One Back) at Screening Visit 2 relative to Screening Visit 1 (improvement of ≥1 standard deviation relative to Screening Visit 1 for either composite measure) as assessed by Cogstate.
3. Meets criteria for dementia (major neurocognitive disorder) due to Parkinson's disease according to criteria of the Diagnostic and Statistical Manual of Mental Disorders, Fifth Edition [APA 2013].
4. Diagnosis of significant central nervous system disease, other than Parkinson's disease, that may affect cognition or the ability to complete the study, including but not limited to other dementias (e.g., Alzheimer's disease, and Huntington's disease).
5. Atypical or secondary parkinsonism due to drugs (e.g., antipsychotics) or disease (such as progressive supranuclear palsy), essential tremor, or multiple system atrophy (e.g., striatonigral degeneration, olivopontocerebellar atrophy), or postencephalitic parkinsonism.
6. History of significant cerebrovascular impairment such as: History in the last 12 months of transient ischemic attacks with structural abnormality or ischemic stroke within 12 months prior to screening, any history of intracerebral hemorrhage due to hypertension, or hypertensive encephalopathy.
7. Lifetime diagnosis of bipolar disorder, schizophrenia, other primary psychotic disorder, or, other psychiatric disorder that would interfere with study participation, in the investigator's opinion. NOTE: Current symptoms of anxiety or depression with a stable treatment of anxiolytics or antidepressants, respectively, are not exclusionary.
8. GDS-SF score of >8 at Screening Visit 1.
9. Poorly controlled psychosis (hallucinations or delusions) that, in the opinion of the Investigator, would interfere with the subject's ability to be compliant with the study protocol.
10. Any other psychiatric disorder that, in the judgment of the Investigator, would interfere with compliance with the study protocol.
11. Meets the criteria for suicidal intent, plan, and/or behavior on the Sheehan Suicidality Tracking Scale (S-STS) at screening or Day 1 by scoring 3 or 4 on Questions 2 or 13, or 2 or higher on any Question 1a (only if 1b is coded YES), 3, 4, 5, 6, 7, 8, 9, 10, 11, 12, or 14.
12. Known history of substance abuse within 1 year prior to first dose of study drug (drug categories defined by the Diagnostic and Statistical Manual of Mental Disorders, Fifth Edition [APA 2013]) and/or substance dependence within 1 year prior to first dose of study drug, not including caffeine and nicotine.
13. Current implantable intracranial stimulator or history of intracranial ablation surgery (e.g., subthalamic, globus pallidus-internal segment).
14. Repetitive transcranial magnetic stimulation within 3 months prior to Screening Visit 1.
15. Use of cholinesterase inhibitors within 2 weeks prior to Screening Visit 1, memantine or amantadine within 3 months prior Screening Visit 1, or anticholinergic medications (e.g., muscarinic antagonist drugs for treatment of Parkinson's disease, including trihexyphenidyl, benztropine, orphenadrine, procyclidine, and biperiden, certain drugs typically prescribed for overactive bladder and urinary incontinence such as solifenacine, oxybutynin, belladonna alkaloids) within 2 weeks prior to Screening Visit 1 or during the study.
16. Use of monoamine oxidase-B inhibitors within 3 months prior to Screening Visit 1 or during the study.
17. Use of digital/application-based cognitive enhancement/brain exercise tools (e.g., Lumosity™) within 3 months prior Screening Visit 1 or during the study.
18. Use of antidepressants with potential impact on cognitive function such as: nefazodone, trazodone, irreversible monoamine oxidase inhibitors, tricyclic antidepressants, noradrenergic and specific serotonergic antidepressant (NaSSA) mirtazapine, or serotonin modulator and stimulator antidepressants such as vilazodone or vortioxetine within 4 weeks prior to Screening Visit 1 or during the study. Unstable antidepressant use (i.e., change in daily dose) within 4 weeks prior to Screening Visit 1 or during the study. Initiation of current therapy with selective serotonin reuptake inhibitors (SSRIs), serotonin and norepinephrine reuptake inhibitors (SNRIs), bupropion, or St. John's Wort within 8 weeks prior to Screening Visit 1. NOTE: Subjects on stable treatment (≥90 days prior to screening) with SSRIs, SNRIs, bupropion, or St. John's Wort may continue this therapy during the study with no expected change in dose. The daily dose of SSRIs or SNRIs should not exceed 20 mg paroxetine, 100 mg sertraline, 20 mg fluoxetine, 10 mg escitalopram, 20 mg citalopram, 100 mg venlafaxine, 50 mg desvenlafaxine, or 60 mg duloxetine.
19. Use of medications with primarily central nervous system activity (e.g., anticonvulsant drugs [including gabapentinoids or pregabalinoids], psychostimulants, benzodiazepines, methylphenidate, barbiturates, first-generation sedating H1 antihistamines, eszopiclone, zolpidem extended-release or any other sedative-hypnotic medications within 2 weeks prior to Screening Visit 1 or during the study. NOTE: Episodic use of certain non-benzodiazepine sleep inducers [e.g., episodic zolpidem immediate-release (single dose at bedtime), zaleplon (single dose at bedtime), ramelteon, or melatonin] are allowed.
20. Any history or current use of antipsychotics for bipolar disorder or schizophrenia, or any use of antipsychotics within 6 months prior to Screening Visit 1 or during the study. Clozapine (up to 25 mg), quetiapine (up to 75 mg), and pimavanserin treatment initiated at least 8 weeks prior to first dose of study drug to address drug-induced or disease-related psychosis is allowed. Participants must be on stable dosage for at least 6 weeks prior to first dose of study drug and expected to continue at the same dose during the study.
21. Participation in experimental treatments for any aspects of Parkinson's disease (motor symptoms, mood symptoms, cognitive symptoms, psycho-behavioral symptoms) in the past 3 months (or 5 half-lives, whichever is longer for a drug product) prior to the first dose of study drug, or in the past 12 months prior to the first dose of study drug with anti-alpha synuclein experimental monoclonal antibody treatment. NOTE: If subject was on placebo arm of disease-modifying treatments, participation is not exclusionary.
22. Use of N-methyl-D-aspartate receptor (NMDAR)-binding drugs (e.g., ketamine, dextromethorphan, methadone, lamotrigine, or esketamine) within 60 days prior to Screening Visit 1 or during the study.
23. Use of recreational or medical marijuana within 30 days prior to the first dose of study drug or during the study. Cannabidiol oil may be used provided that the dose is stable, and the urine drug screen results are negative on Day 1.
24. Positive screen for illicit drugs of abuse, including phencyclidine, barbiturates, benzodiazepines, opiates, methadone, cocaine, cannabinoids, and amphetamines. Subjects who have a positive result at screening for appropriate use of prescribed medication(s) must have a negative result at Day 1.
25. Uncontrolled Type I or Type II diabetes mellitus (hemoglobin A1c >8%) or uncontrolled hypertension.
26. Body mass index >35 kg/m² at screening.
27. Estimated creatinine clearance <30 mL/minute/SA at screening (as calculated by the central laboratory) or history of clinically significant renal disease, as assessed by the Investigator or the Sponsor-designated medical monitor.
28. Evidence of folic acid deficiency (indicated by folate level below lower limit of normal) or B12 deficiency (indicated by methylmalonic acid levels above the upper limit of normal). NOTE: subjects may be rescreened if there is no improvement in cognition after 3 months of adequate treatment for folic acid or vitamin B12 deficiency.
29. History of allergy, sensitivity, or intolerance to NMDAR ligands, including ketamine, esketamine, dextromethorphan, memantine, methadone, dextropropoxyphene, or ketobemidone, as well as current use of such agents.
30. Screening QT interval corrected for heart rate by Fridericia's formula >450 msec (males) or >470 msec (females) or an electrocardiogram that is not suitable for QT measurements (e.g., poorly defined termination of T-wave in the Investigator's opinion).
31. Known familial history or known presence of long QT syndrome, or a known history of past or current clinically significant arrhythmias or ischemic heart disease.
32. Known diagnosis of a current infectious disease, including human immunodeficiency virus infection, hepatitis, or other ongoing infectious disease that the Investigator considers clinically significant.
33. Current evidence of dysplasia or history of malignancy (including lymphoma and leukemia) in the last 5 years, except for successfully treated non-metastatic basal cell or squamous cell carcinoma of the skin or localized carcinoma in situ of the cervix.
34. History of gastrointestinal disease or surgery (except simple appendectomy or hernia repair), leading to impaired drug absorption.
35. Abnormal laboratory results, electrocardiogram results, medical history, or concurrent conditions which, in the opinion of the Investigator or Sponsor, would preclude safe study participation or interfere with study procedures/assessments.
36. Uncorrected hypothyroidism or hyperthyroidism. NOTE: Subjects with compensated hypothyroidism with normal thyroid-stimulating hormone levels may be enrolled.
37. Impaired hepatic function characterized by a previous known diagnosis of chronic liver disease and/or the presence of direct bilirubin, alanine aminotranferase, aspartate aminotransferase, alkaline phosphatase, or gamma-glutamyl transferase >1.5×the upper limit of normal at screening.

### Test Product, Dose, and Mode of Administration:

The Compound 10 mg, 30 mg, and 100 mg oral capsules, 1 capsule once daily by mouth without food. Subjects should not consume food for 2 hours before each dose or 1 hour after each dose of study drug.

### Reference Therapy, Dose, and Mode of Administration:

Placebo, oral capsules, 1 capsule once daily by mouth without food. Subjects should not consume food for 2 hours before each dose or 1 hour after each dose of study drug.

### Duration of Study Participation:

Screening: 14 to 28 days
Treatment: 12 weeks of blinded treatment with either the Compound or placebo
Follow-Up: 14 days

### Criteria for Evaluation:

### Safety:

Rates of adverse events and serious adverse events
Change from baseline in physical examinations
Change from baseline in vital signs, clinical laboratory values, and electrocardiogram results
Change from baseline in dissociative effects, psychosis, and hallucinatory symptoms as measured by the Neuropsychiatric Inventory (NPI-12)
Change from baseline in suicidal ideation and behavior as measured by the S-STS

### Exploratory:

Change in composite z-score based on DSST, Category Fluency Test Two Back Test, One Back Tests, Identification Test, and Groton Maze Learning Tests of Executive Function and Attention/Working Memory
Change in Executive Functions composite score (assessed by Groton Maze Learning Test, Two Back Test and Category Fluency Test)
Change in Attention/Working Memory composite score (assessed by DSST, One Back Test, and Identification Test)
Change in Memory composite score (assessed by International Shopping List and Continuous Paired Associate)
Change in individual performance based neuropsychological test scores
Clinical global change as measured by the Clinician's Interview-Based Impression of Change-Plus Caregiver Input (CIBIC-Plus)
Change from baseline in sleep quality as measured by the Scales for Outcomes in Parkinson's Disease Sleep Disturbances (SCOPA Sleep)
Change in cognition as measured by the MoCA
Change in Visuospatial Functions (as measured by Benson Complex Figure and Line Orientation tests)
Change in daily functions as measured by the 12-item version of Everyday Cognition (ECog12)
Change from baseline in non-motor symptoms associated with Parkinson's disease as measured by MDS-UPDRS Part 1
Change from baseline in motor symptoms and various motor symptom profiles (e.g., akinetic versus tremor-dominant Parkinson's disease) as measured by the MDS-UPDRS Part 3
Change from baseline in motor complications as measured by the MDS-UPDRS Part 4
Change from baseline in mood, anhedonia, and apathy as measured by the NPI-12 apathy/indifference subscore and Item 7 of the Movement Disorder Society Unified Parkinson's Disease Rating Scale (MDS-UPDRS)
Plasma concentrations of the Compound
Potential relationships between COMT and APOE polymorphisms, symptoms, and efficacy
Potential relationships between pharmacogenomics and efficacy
   Sample Size: The planned sample size is approximately 135 randomized subjects.

### Statistical Methods:

Descriptive statistics for categorical variables will include the number and percentage of subjects with each characteristic. Percentages will be based on the number of subjects with non-missing values. Descriptive statistics for ordinal (e.g. Likert scale) and continuous variables will include the number of subjects with non-missing values, mean, median, standard deviation, minimum value, and maximum value.

Safety analyses will be based on the Safety Population, defined as all subjects who receive at least one dose of study drug. Efficacy analyses will be based on the modified Intent-to-Treat Population, defined as all subjects in the Safety Population with at least one (1) postbaseline assessment of efficacy.

For each efficacy endpoint, change from baseline to each applicable post-baseline visit will be assessed for treatment group differences using a mixed model for repeated measures (MMRM) with factors for study site, treatment, week, and the treatment-by-week interaction, and using the baseline response variable value as a covariate.

As a sensitivity analysis, an analysis of covariance (ANCOVA) with fixed factors for study site and treatment, with baseline value as a covariate will be used.

Adverse events will be categorized by system organ class and preferred term with the Medical Dictionary for Regulatory Activities (MedDRA). Summary tables for treatment-emergent adverse events (TEAEs) will include number and percent of subjects experiencing TEAEs by system organ class and preferred term.

Mean change in clinical laboratory and vital signs from baseline to each post-baseline visit will be summarized descriptively. Clinical laboratory results considered clinically important by the Investigator will be identified.

The planned statistical analysis methods will be described in more detail in the Statistical Analysis Plan, which will be finalized prior to database lock.

Dosing and Administration: There will be 4 treatment groups:
10 mg of the Compound by mouth once daily for 12 weeks
30 mg of the Compound by mouth once daily for 12 weeks
100 mg of the Compound by mouth once daily for 12 weeks
Placebo by mouth once daily for 12 weeks

### Study Drug Description, Appearance, Packaging, and Labeling:

The Compound is a small molecule, (S) 2-(4-methoxybenzyl)-2,7-diazaspiro[3.5]nonane-1,6-dione, that will be provided as capsules for oral administration in strengths of 10, 30, and 100 mg of the Compound per capsule. Matching placebo capsules will also be provided.

The oral formulation of the Compound is composed of inert United States Pharmacopeia and National Formulary-grade (USP-NF) excipients in a capsule; the formulation comprises a dry blend of the Compound with microcrystalline cellulose USP-NF, pregelatinized starch USP-NF, magnesium stearate USP-NF, and filled into hydroxypropyl methylcellulose capsules.

Matching placebo capsules will contain only the inactive ingredients listed previously.

The study drug will be provided in bottles. The labels will include “the Compound Oral Capsules or Placebo to Match”, capsule count, bottle number, storage conditions, Sponsor name, and investigational use statement. Each bottle of study drug will include 32 capsules of either 10 mg, 30 mg, and 100 mg of the Compound or matching placebo capsules according to the randomization schedule.

### Example 3: A Study to Evaluate the Compound in Subjects with Mild Cognitive Impairment Associated with Parkinson's Disease

This study is similar to Example 2 except that only one test dose is studied, which test dose is 30 mg of the Compound.

In addition, for inclusion criteria the patient has a Montreal Cognitive Assessment Score of 15-25, and verifiable impairment as defined by a CGI-S score of at least 3 before administration. Patients before administration can have mild cognitive impairment or mild dementia. In addition, patients before administration can have possible mild dementia with Lewy bodies (DLB) and identified cognitive deficits, according to current consensus criteria, and by extension mild cognitive impairment based on criteria for probable dementia with Lewy bodies.

In addition, the study also adds for exclusion criteria any patients with History of severe infection with COVID-19 requiring hospitalization, treatment with oxygen or mechanical ventilation, that may interfere with study participation, as assessed the investigator, and any subject with a medical history of COVID-19 infection (positive test) within the last two (2) months, or current symptoms consistent with COVID-19 infection (not tested), e.g. loss of smell, sore throat, cough or fever (2 or more symptoms at the same time), as assessed by the investigator.

In addition, this study adds a set of efficacy criteria as follows: Primary:
Change in z-scores of Category Fluency Test, Two Back Test, One Back Tests, Identification Test, and Groton Maze Learning Tests of Executive Function and Attention/Working Memory

### Secondary:

Clinical global change as measured by the Clinician's Interview-Based Impression of Change-Plus Caregiver Input (CIBIC-Plus)
Change in the PDAQ-15, 15-item Penn Parkinson's Disease Activities Questionnaire.
Change in daily functions as measured by the 12-item version of Everyday Cognition (ECog12)

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating cognitive impairment associated with neurodegenerative disease in a patient in need thereof comprising:
administering daily to the patient an effective amount of a compound selected from (S) 2-(4-methoxybenzyl)-2,7-diazaspiro[3.5]nonane-1,6-dione and a pharmaceutically acceptable salt thereof.

2. The method of claim 1, wherein the neurodegenerative disease is Parkinson's disease or Alzheimer's disease.

3. The method of claim 1, comprising orally administering about 10 mg, about 30 mg or about 100 mg of the compound daily.

4. The method of claim 1, wherein the patient has a Montreal Cognitive Assessment Score before administration of 15-25 or 17 or more.

5. The method of claim 1, wherein the patient has cognitive impairment in at least one of the following cognitive domains: attention, mental processing speed, executive functioning/problem solving, and visuospatial function.

6. The method of claim 1, wherein the patient has cognitive impairment as defined by scoring below or about 1.5 standard deviations of age and education based norms in a least one of executive function or attention/working memory cognitive domains, using one or more of: Movement Disorder Society MCI-PD diagnostic criteria, Groton Maze Learning Test, Digit Symbol Substitution Test (DSST), Cogstate One Back test, Cogstate Identification Test or the Stroop test, before administration of the compound.

7. The method of claim 1, wherein the patient has dementia related psychosis.

8. A method of treating cognitive impairment associated with Parkinson's disease in a patient having a Montreal Cognitive Assessment of 17 or more, comprising administering daily to the patient an effective amount of a compound selected from (S) 2-(4-methoxybenzyl)-2,7-diazaspiro[3.5]nonane-1,6-dione and a pharmaceutically acceptable salt thereof.

9. The method of claim 1, wherein the patient is currently being administered levodopa and/or a levodopa enhancer.

10. The method of claim 1, wherein, after 12 weeks or more of daily administration of the compound, the patient has improved or stabilized cognition as measured by one or more of: improvement in a composite z-score based on DSST, Category Fluency Test Two Back Test, One Back Tests, Identification Test, and Groton Maze Learning Tests of Executive Function and Attention/Working Memory.

11. The method of claim 1, wherein, after daily administration of the compound for 12 weeks or more, the patient shows improvement in an Executive Functions composite score as assessed by one or more of Groton Maze Learning Test, Two Back Test and Category Fluency Test, as compared to baseline.

12. The method of claim 1, wherein, after daily administration of the compound for 12 weeks or more, the patient has improvement in an Attention/Working Memory composite score as assessed by one or more of DSST, One Back Test, and Identification Test.

13. The method of claim 1, wherein, after daily administration of the compound for 12 weeks or more, the patient has memory improvement as measured by a Memory composite score using an International Shopping List or Continuous Paired Associate tests.

14. The method of claim 1, wherein, after daily administration of the compound for 12 weeks or more, the patient has improved sleep quality as measured by the Scales for Outcomes in Parkinson's Disease Sleep Disturbances (SCOPA Sleep) compared to baseline.

15. The method of claim 1, wherein, after 12 weeks or more of daily administration of the compound, the patient has the same or better cognition as measured by the MoCA.

16. The method of claim 1, wherein, after 12 weeks or more of daily administration of the compound, the patient has improvement in daily functions as measured by the 12-item version of Everyday Cognition (ECog12).

17. The method of claim 1, wherein, after 12 weeks or more of daily administration of the compound, the patient has improvement as measured by the 12-item version of Penn Parkinson's Disease Activities Questionnaire (PDAQ-15)).

18. The method of claim 1, wherein the patient has a score of at least 3 on the Clinical Global Impression Scale (CGI-S) for mental illness.

19.-20. (canceled)

21. A method of treating cognitive impairment associated with neurodegenerative disease in a patient in need thereof comprising:
administering daily to the patient an effective amount of a pharmaceutical formulation comprising a compound selected from (S) 2-(4-methoxybenzyl)-2,7-diazaspiro[3.5]nonane-1,6-dione and a pharmaceutically acceptable salt thereof; and a pharmaceutically acceptable excipient.

22.-24. (canceled)

25. The method of claim 21, comprising orally administering about 10 mg, about 30 mg, or about 100 mg of the compound.

26. (canceled)
